(19) United States
(12) Patent Application Publication
(10) Pub. No.: US 2023/0333045 A1
(43) Pub. Date: Oct. 19, 2023

(54) HIGHLY CHEMICALLY STABLE APTAMER SENSORS

(71) Applicant: University Of Cincinnati, Cincinnati, OH (US)

(72) Inventors: Ryan Jeffrey White, Cincinnati, OH (US); Jason Charles Heikenfeld, Cincinnati, OH (US)

(21) Appl. No.: 18/027,392

(22) PCT Filed: Sep. 24, 2021
(86) PCT No.: PCT/US2021/051869

§ 371 Date: Mar. 21, 2023

Related U.S. Application Data
(60) Provisional application No. 63/150,663, filed on Feb. 18, 2021.
(60) Provisional application No. 63/082,825, filed on Sep. 24, 2020.

(51) Int. Cl. G01N 27/327; A61B 5/1468; G01N 33/543; G01N 33/53; G01N 27/30; G01N 27/333

(52) U.S. Cl. CPC G01N 27/3277; A61B 5/1468; G01N 33/5438; G01N 33/5308; G01N 27/308; G01N 27/333

(57) ABSTRACT

A sensing electrode is provided. The sensing electrode (104) includes a sensor substrate (120), a recognition element (106) tethered to the sensor substrate, and an encasement (108) encasing the sensor substrate. A device including the sensing electrode is also provided. In certain embodiments, the substrate is not gold, and the tethering is with a non-thiol bond.

### CROSS-REFERENCE TO RELATED APPLICATIONS

This application claims priority to, and the benefit of the filing date of, U.S. Provisional Application No. 63/082,825 filed Sep. 24, 2020 and U.S. Provisional Application No. 63/150,663 filed Feb. 18, 2021, the disclosures of which are incorporated by reference herein in their entirety.

### TECHNICAL FIELD

The present invention relates to the use of electrochemical, aptamer-based (E-AB) sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Aptamers are nucleic acid or peptide molecules that bind to a target molecule with high specificity. After selection and enrichment, aptamers possess similar affinities to antibody-antigen pairs, but have the advantage of being able to be synthesized using standard methods. As synthetic molecules, aptamers also have unique advantages in the control of their size and their amenability for chemical modification and, as such, have been widely developed and applied in the development of sensors. Electrochemical, aptamer-based (EAB) sensors have emerged in recent years as a platform to detect proteins, small molecules, and inorganic ions, relying on the induced conformational change of oligonucleotide aptamers in the presence of specific analyte. When a target molecule binds to an aptamer, which is tethered to an electrode surface, changes in the aptamer structure are measured by changes in the electrochemical signal of an attached redox label on the aptamer. EAB sensor technology presents a stable, reliable, bioelectric sensor that is sensitive to the target analyte in a sample, while being capable of multiple analyte capture events during the sensor lifespan.

The use of EAB sensors to monitor in vivo analyte levels has been suggested. However, as with other types of sensors, EAB sensors are subject to fouling after prolonged exposure to whole blood and other complex samples, making this approach difficult. In addition, prolonged-use EAB sensors have many other challenges to overcome, including stability, aptamer degradation and useful life of the unit. Therefore, a need still exists for improved EAB sensors that can be used in vivo for extended periods of time.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Aspects of the present invention reduce and/or eliminate/overcome the current drawbacks with prolonged use of EAB sensors. To do this, one aspect of the present invention provides a device configured and/or including features to allow its prolonged use without problems with fouling, stability, aptamer degradation, or useful life of the unit.

To that end, in one embodiment, the device includes a sensing electrode. The sensing electrode includes a sensor substrate, a recognition element tethered to the sensor substrate, and an encasement encasing the sensor substrate. In various specific embodiments, the encasement includes at least one of a dialysis membrane, an osmosis membrane permeable to ions and impermeable to small molecules and proteins, a water permeable membrane, or a combination thereof.

In another embodiment, the sensing electrode includes (1) a sensor substrate, and (2) a recognition element based on an aptamer with an attached redox tag. The recognition element is tethered to the sensing electrode. In this embodiment, the substrate is not gold, and the tethering is with a non-thiol bond.

### BRIEF DESCRIPTION OF THE DRAWINGS

The objects and advantages of the disclosed invention will be further appreciated in light of the following detailed descriptions and drawings in which:

FIG. 1 is a cross-sectional view of a sensing electrode according to an embodiment of the disclosed invention.

FIG. 2 is a cross-sectional view of a device including the sensing electrode according to an embodiment of the disclosed invention.

FIG. 3 is a cross sectional view of a sensing electrode according to an embodiment of the disclosed invention in a fluid sample.

### DETAILED DESCRIPTION OF THE INVENTION

### Definitions

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

As used herein, the term “sensing monolayer” means aptamers that are functionalized with a redox tag, such as methylene blue or other redox tag, and attached onto an electrode such as gold by thiol linkage or other suitable chemistry, and the space in between the aptamers on the electrode passivated by a passivating material such as mercaptohexanol or other suitable passivating material.

The devices and methods described herein encompass the use of sensors. A sensor, as used herein, is a device that is capable of measuring the concentration of a target analyte in solution. In one embodiment, the sensor uses a recognition element, linked to the sensor surface, to identify a target analyte. The target analyte may be any inorganic or organic molecule, for example: a small molecule drug, a metabolite, a hormone, a peptide, a protein, a carbohydrate, a nucleic acid, or any other composition of matter. The target analyte may comprise a drug. The drug may be of any type, for example, including drugs for the treatment of cardiac system, the treatment of the central nervous system, that modulate the immune system, that modulate the endocrine system, an antibiotic agent, a chemotherapeutic drug, or an illicit drug. The target analyte may comprise a naturally-occurring factor, for example a hormone, metabolite, growth factor, neurotransmitter, etc. The target analyte may comprise any other species of interest, for example, species such as pathogens (including pathogen induced or derived factors), nutrients, and pollutants.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

As described above, current EAB sensors and devices include problems with fouling, stability, aptamer degradation, and useful life of the unit. The present invention addresses these issues by providing a variety of improvements to EAB sensors that prolong the use of the unit. EAB sensors comprise one or more working electrodes to which recognition elements functionalized with redox indicators are bound. The one or more electrodes may comprise various materials and configurations. The electrode may comprise any suitable electrode material for electrochemical sensing, including, for example: gold or any gold-coated metal or material, titanium, tungsten, platinum, carbon, aluminum, copper, palladium, mercury films, silver, oxide-coated metals, semiconductors, graphite, carbon nanotubes, and any other conductive material upon which biomolecules can be conjugated.

To that end, in one embodiment, the device includes a sensing electrode. The sensing electrode includes a sensor substrate, a recognition element tethered to the sensor substrate, and an encasement encasing the sensor substrate. In various specific embodiments, the encasement includes at least one of a dialysis membrane, an osmosis membrane permeable to ions and impermeable to small molecules and proteins, a water permeable membrane, or a combination thereof.

In another embodiment, the sensing electrode includes (1) a sensor substrate, and (2) a recognition element based on an aptamer with an attached redox tag. The recognition element is tethered to the sensing electrode. In this embodiment, the substrate is not gold, and the tethering is with a non-thiol bond.

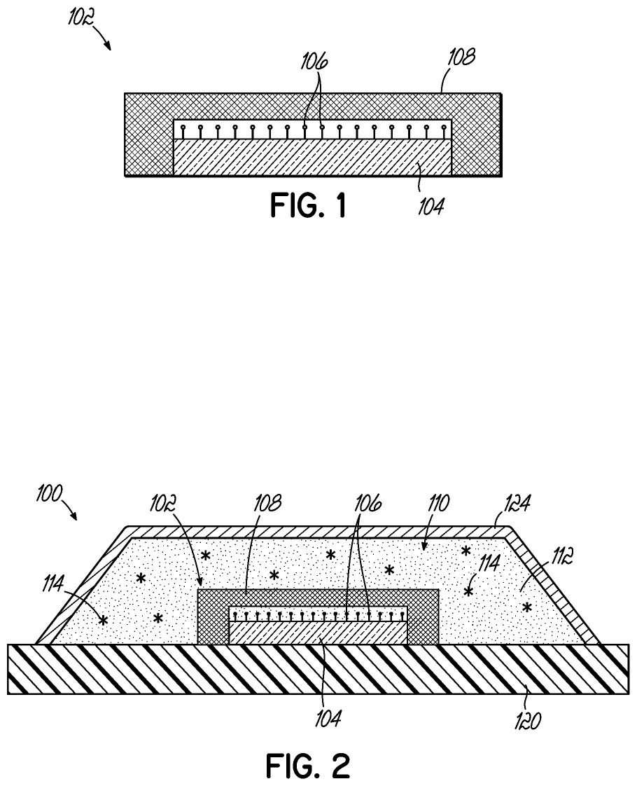

More specifically, and with reference to FIG. 1, a cross-section of a sensing electrode 102 is shown. The sensing electrode 104 includes recognition elements 106, and a filtration element 108. The sensing electrode 104 may be carried by a substrate 120 and is configured to detect an analyte 112 (shown in FIG. 2) in a fluid sample 110 (shown in FIG. 2). More specifically, the sensing electrode 104 is configured to measure a concentration of a target analyte 112 in a fluid sample 110. In one embodiment, the sensing electrode 104 may be included in a device 100 (shown in FIG. 2) such as, an indwelling device, ranging from partially indwelling (e.g., a needle) to fully indwelling (e.g., a capsule implanted in the body or skin). The device 100 includes a large filtration membrane and/or a device encasement 124 (shown in FIG. 2) to keep out cellular and other large debris. Device encasements 124 are described in more detail below.

As previously described, the sensing electrode 104 includes a sensor substrate 120. The sensor substrate 120 includes a sensing surface, and recognition elements 106 are tethered to the sensing surface of the sensing electrode 120. The sensor substrate 120 may be any suitable material configured to support the device 100. In some embodiments, the sensing electrode 104 includes carbon atoms that form, or are configured to form, covalent carbon-carbon bonds with the recognition elements 106, and accordingly tether, or are configured to tether, the recognition elements 106 to the sensing electrode 104. In an embodiment, the sensor electrode 104 includes diamond. In an embodiment, the sensing electrode 104 surface includes diamond, and the diamond included on the sensing electrode 104 surface is included in a carbon-carbon covalent bond with the recognition element 106.

As previously described, the sensing electrode 104 further includes a recognition element 106. The recognition element 106 is tethered to, or configured to be tethered to, the sensing electrode 104. In some examples, the recognition element 106 is an aptamer or a plurality of aptamers tethered to the sensing electrode 104. The aptamers 106 may further include a redox tag such as methylene blue. The tether of the recognition element 106 to the sensing electrode 104 surface is a covalent bond. In some examples, the covalent bond is a carbon-carbon covalent bond. In one embodiment, a recognition element is covalently linked by a carbon-carbon bond to a sensing surface as opposed to the typical Au-thiol (gold-thiol) chemisorbed bond. While Au-thiol bonds are covalent-like, Au-thiol have a bond strength of about 50-100 kJ mol⁻¹. Furthermore, conventional Au-thiol bonds are prone to reductive or oxidative desorption.

The covalent carbon-carbon bond of the present invention is much more stable. In one embodiment, the recognition element 106 is linked to the sensing electrode 104 surface with a bond strength of at least about 200 kJ mol⁻¹. In another embodiment, the recognition element 106 is linked to the sensing electrode 104 surface with a bond strength of at least about 300 kJ mol⁻¹. In another embodiment, the recognition element 106 is linked to the sensor substrate surface with a bond strength of at least about 400 kJ mol⁻¹. In one embodiment, the sensing electrode 104 surface comprises indium tin oxide (ITO) or another suitable electrode material that creates, or is configured to create, a covalent bond with the recognition element 106. In another embodiment, the duration over which the bond between the sensing electrode 104 surface and the recognition element 106 will stay intact in solution buffer or serum or other test or storage fluid is at least one of 1 week, 2 weeks, 1 month, 2 months, 6 months.

Aptamer degradation can also affect the stability of an EAB sensor. For example, aptamers may degrade due to interaction with reactive oxygen produced via water electrolysis at the gold electrodes. In another example, pH and/or salinity conditions may degrade aptamers. In addition, RNA based elements are prone to nuclease activity which cause further degradation. With continued reference to FIG. 1, in one embodiment, the sensing electrode 102 further includes an encasement 108 over the sensing electrode 102. The encasement 108 may be a membrane such as a dialysis membrane, an osmosis membrane permeable to ions and impermeable to small molecules and proteins, another type of membrane that is at least permeable to water and impermeable to the target analyte 112 (shown in FIG. 2), or a combination thereof. The encasement 108 may contain a concentration of an anti-oxidant species. In another embodiment, the encasement 108 of the present invention enables local control of the pH and/or salinity of the sensor interface with the fluid sample 110 (shown in FIG. 2). In one embodiment, a hydrogel can be used as the encasement 108 to keep larger proteins from degrading the aptamer. In another embodiment, nuclease inhibitors are captured in the encasement 108, resulting in an active form of protection within the encasement 108.

In a particular embodiment, the sensing electrode 102 is an electrochemical aptamer-based sensor for measuring the concentration of a target analyte 112 in a fluid sample 110. The sensing electrode 102 includes a sensing electrode 104 and one or more recognition elements 106, such as aptamers, bound to a carbon substrate. The sensing electrode 102 further includes an encasement 108. In an embodiment, the encasement 108 is selected from the group consisting of porous membranes, other encasements, or combinations thereof. Further, the sensor substrate 102 may be encased within the encasement 108, which allows the fluid sample 110 to contact the sensing electrode while preventing contact between the sensing electrode and fouling species present in the sample. In another embodiment, the carbon substrate is diamond.

In one embodiment, the one or more aptamers are bound to the carbon substrate with a bond strength of at least about 200 kJ mol⁻¹. In another embodiment, the one or more aptamers are bound to the carbon substrate with a bond strength of at least about 300 kJ mol⁻¹. In another embodiment, the one or more aptamers are bound to the carbon substrate with a bond strength of at least about 400 kJ mol⁻¹.

Sensing electrodes 104 with aptamers 106 may also make use of a blocking layer such as mercaptohexanol or hexane thiol on gold electrodes, but again thiol bonding is a weaker bonding mechanism that the non-gold bonding mechanisms of the present invention. Therefore, the blocking layer based on hexanol, hexane, or other suitable molecules may, like aptamer 106, also include linkage chemistry for carbon, diamond, or other suitable electrodes 104.

Carbon electrodes must be functionalized prior to aptamer attachment. Functionalization is performed by grafting 4-azidobenzene diazonium via a single potential pulse, holding potential at or below the peak potential for reduction of the diazonium salt. The application of this short time frame pulse presumably limits surface passivation that can occur from a more densely packed surface layer of azidobenzene molecules or from multilayer formation. After functionalization, the azide groups grafted on the surface can be coupled to 5′-hexyne-terminated aptamers in the presence of a Cu⁺ catalyst using standard click chemistry. The result is covalent attachment of the aptamers to the carbon surface via a stable five-member nitrogen-carbon heterocycle. Successful sensor development can be accomplished via control of the number of chemical handles grafted to the electrode surface, which is achieved by the short potential pulses (ms-s) applied. While similar chemistry as to that now described has been used in the past to couple DNA to carbon, such techniques have not been tried for the purpose of structure-switching aptamer-based sensors. A combination of short pulse grafting of the diazonium, as disclosed by the present inventors, may provide a more suitable surface for the development of aptamer sensors at least because less passivation is present in such embodiments. Accordingly, through a combination of the grafting and chemistry techniques described herein, an improvement to aptamer sensor performance may be shown. There may be a potential dependent deposition of diazonium to prevent multilayer formation, however, time has been explored as another variable for more control with regard to the current invention.

One way to increase stability is to use C═C double bonds. The other is to continue to use gold. For example, passivating the surface to fill in space, covering with membranes. Encapsulated system, as stuff comes off, excess aptamer can fill in the space. Keep some discussion of the gold surface in the application specifically with the encasement embodiment.

With reference to FIG. 2, a cross-section of a device 100 including the sensing electrode 102 is shown. The device 100 is shown having a fluid sample 110 within the device 100, however, the fluid sample 110 is not a part of the device 100, itself, but rather is intended as a workpiece sample that the device 100 may operate upon. Accordingly, the device 100 is configured to hold a fluid sample 110 proximately to the sensing electrode 102, and the device includes a device encasement 124, a device substrate 120, and the sensing electrode 102, as described above.

The device encasement 124 is a barrier that prevents the fluid sample 110 from spilling or leaking into the surrounding environment. The device encasement 124 may be made of any material suitable for containing the fluid sample 110 within the device 100, and proximate to the sensing electrode 102. The device encasement 124 is coupled to the device substrate 120. Similar to the device encasement 124, the device substrate 120 prevents the fluid sample 110 from spilling or leaking the in the environment surrounding the device 100. In addition, in an embodiment, the device substrate 120 is coupled to the sensing electrode 102, and holds the sensing electrode 102 in place. The device encasement 124 is coupled to the device substrate 120, and together, the device encasement and the device substrate form a housing configured to house the fluid sample 110.

The device 100 may in configured for in vivo or ex vivo use, and the sample fluid upon which the sensing electrode 102 may be configured to operate may be selected from a group consisting of bodily fluids such as blood, interstitial fluid, sweat, urine, river water, or other suitable sample fluid. As shown in FIG. 2, the fluid sample 110 includes an analyte 112 that the sensing electrode 102 is configured to measure. The encasement 108 includes pores sized to allow the analyte to permeate therethrough. In addition, the recognition element 106 is configured to adsorb and/or chemically react with the analyte 112 after the analyte 112 has permeated the encasement 108. In an embodiment, the recognition element 106 is specifically configured to be selective toward the analyte 112. The sensing electrode 102 detects the adsorption and/or chemical reaction of the analyte 112 with the recognition element, and accordingly, the sensing electrode 102 may determine the concentration of the analyte 112 in the fluid sample 110.

With continued reference to FIG. 2, a fouling species 114 may be included in the fluid sample 110. The fouling species 114, if allowed to contact the recognition elements 106 may cause false readings of the concentration of the analyte 112 in the fluid sample 110. Alternatively, or in addition, the fouling species 114 may cause deterioration, decreased sensitivity, or otherwise damage the recognition element 106 or the sensing electrode 102 if the fouling species 114 contacts the recognition element 106. The encasement 108 is configured to prevent the fouling species 114 from permeating therethrough and contacting the recognition element 106. For example, the encasement 108 may include pores sized to prevent the fouling species 114 from permeating therethrough. As described above, the fluid sample 110 including the analyte 112 and the fouling species 114 is included in FIG. 2 as a workpiece example, and not as a component of the device 100 or the sensing electrode 102, generally.

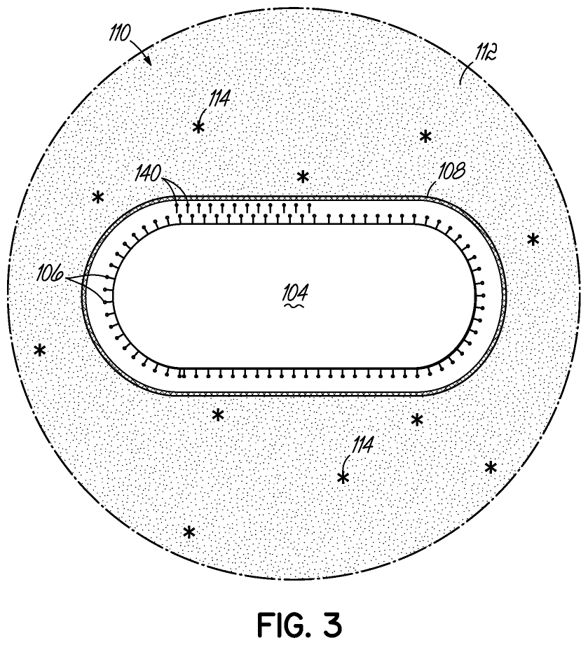

Referring to FIG. 3, an alternative embodiment of the sensing electrode 102 is shown. Particularly, FIG. 3 shows a cross section of a capsule immersed in an in vivo fluid sample 110. As with the sensing electrode 102 shown in FIG. 1, the sensing electrode 102 shown in FIG. 3 includes recognition elements 106, an encasement 108, and a sensing electrode 104. The embodiment shown in FIG. 3 further includes the encasement 108 containing, or configured to contain, additional non-bonded recognition elements 140. The non-bonded recognition elements 140 are untethered to the sensing electrode 104. In use, these additional, non-bonded recognition elements 140 may replace recognition elements 106 that have degraded or fallen off the sensing electrode 104 during use. The non-bonded recognition elements 140 rebond on the surface of the sensing electrode 104, which may include gold particles or carbon, for example that are revealed as degraded recognition elements 140 debond. In one embodiment, the encasement 108 seals the non-bonded recognition elements 140, the sensing electrode 104, and the recognition elements 106 tethered to the sensing electrode 104. In an embodiment, the encasement 108 has a pore size that correlates with the size of one or more target analyte(s) 112, but the pore size is too small for additional non-bonded recognition elements 140. In an embodiment, at least due to the aforementioned configuration, the non-bonded recognition elements 140 are contained in the encasement 108 in a solution adjacent to the sensing electrode 102.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A sensing electrode comprising:
a sensor substrate; and
a recognition element based on an aptamer with an attached redox tag, the recognition element tethered to the sensing electrode;
wherein the substrate is not gold and the tethering is with a non-thiol bond.

2. The sensing electrode of claim 1, wherein the recognition element is tethered to the sensor substrate by a carbon-carbon covalent bond.

3. The sensing electrode of claim 1, wherein the sensor substrate comprises carbon.

4. The sensing electrode of claim 1, wherein the sensor substrate comprises diamond.

5. The sensing electrode of claim 1, wherein the recognition element is tethered to the sensor substrate with a bond having a bond strength of at least about 200 kJ mol⁻¹.

6. The sensing electrode of claim 1, wherein the recognition element is tethered to the sensor substrate with a bond having a bond strength of at least about 300 kJ mol⁻¹.

7. The sensing electrode of claim 1, wherein the recognition element is tethered to the sensor substrate with a bond having a bond strength of at least about 400 kJ mol⁻¹.

8. The sensing electrode of claim 1, wherein the sensor substrate comprises indium tin oxide.

9. The sensing electrode of claim 1, further comprising a non-bonded recognition element, the non-bonded recognition element being untethered to the sensor substrate.

10. The sensing electrode of claim 9, further comprising an encasement encasing the substrate.

11. The sensing electrode of claim 10, wherein the encasement comprises at least one of a dialysis membrane, an osmosis membrane permeable to ions and impermeable to small molecules and proteins, a water permeable membrane, or a combination thereof.
